(19) United States
(12) Patent Application Publication
(10) Pub. No.: US 2014/0277347 A1
(43) Pub. Date: Sep. 18, 2014

(54) ENDOPROSTHETIC DEVICE COMPRISING A SUPPORT CHANNEL CAPAPBLE OF RECEIVING A BRANCH ENDOPROSTHETIC DEVICE

(71) Applicant: W. L. GORE & ASSOCIATES, INC., NEWARK, DE (US)

(72) Inventors: John R. Daugherty, Flagstaff, AZ (US); Joshua J. Lovekamp, Flagstaff, AZ (US)

(73) Assignee: W. L. GORE & ASSOCIATES, INC., NEWARK, DE (US)

(21) Appl. No.: 14/190,187

(22) Filed: Feb. 26, 2014

Related U.S. Application Data
(60) Provisional application No. 61/793,171, filed on Mar. 15, 2013.

(51) Int. Cl. A61F 2/856; A61F 2/95

(52) U.S. Cl. CPC A61F 2/856; A61F 2/95
USPC 623/1.11; 623/1.15; 623/1.34; 623/1.13

(57) ABSTRACT

The present disclosure includes an endoprosthesis comprising a tube or substantially tubular lumen. The tube can comprise a first end and a second end, and a side wall can extend between the first and second ends. The side wall can be fenestrated by a side opening or fenestrated portion. The endoprosthetic device can further comprise a patch or support wall coupled to an inner surface of the side wall. The patch can overlap the fenestration. Further, the patch can have a first open edge and/or a second open edge, both of which can open to receive a branch endoprosthetic device. The patch can be movable between a closed configuration adjacent to the inner surface of the side wall and an open configuration spaced apart from the inner surface of the side wall. The open configuration can define a support channel that receives the branch endoprosthetic device.

### FIELD

The present disclosure generally relates to endoprosthetic devices for treating diseases of the vasculature, and more particularly to endoprosthetic devices comprising fenestrations or openings capable of receiving branch endoprosthetic devices.

### BACKGROUND

Many endoprosthetic medical devices (or endoprostheses), such as, for example, stent-grafts, are constructed to reinforce, replace, bridge, or otherwise treat a part of a blood vessel. An endoprosthetic medical device may thus guide blood flow through a lumen defined by a generally tubular interior of such a vessel.

Occasionally, it may be necessary to implant an endoprosthetic device within a main vessel of a patient's body such that the device would, without adaptation, occlude or block one or more side-branch vessels extending from the main vessel. Thus, to permit blood to flow between a main vessel and a side branch vessel, certain fenestrated endoprosthetic devices have been developed. Endoprostheses of this type may be coupled to one or more side branch endoprostheses, so that blood is allowed to flow between a main vessel and a side branch vessel.

To this end, however, prior art endoprosthetic devices have included a variety of complex side branch endoprosthesis receiving portals. Thus, an improved fenestrated endoprosthesis and a method for deploying the same are desirable.

### BRIEF DESCRIPTION OF THE DRAWINGS

The features and advantages of the present disclosure will become more apparent from the detailed description set forth below when taken in conjunction with the drawings, wherein:

FIG. 1 illustrates a perspective view of an endoprosthetic device comprising a fenestration and a patch;

FIG. 2 illustrates a perspective view of an endoprosthetic device coupled to a branch endoprosthetic device in a first configuration;

FIG. 3A illustrates a perspective view of an endoprosthetic device coupled to a branch endoprosthetic device in a second configuration;

FIG. 3B illustrates a perspective view of a patch coupled to an endoprosthetic device;

FIG. 4 illustrates a cross-sectional view of an endoprosthetic device coupled to a branch endoprosthetic device in a first configuration;

FIG. 5 illustrates a cross-sectional view of an endoprosthetic device coupled to a branch endoprosthetic device in a second configuration;

FIG. 6 illustrates a perspective view of an endoprosthetic device having a patch and cannulated by a guidewire;

FIG. 7 illustrates a perspective view of a fenestration and a patch in an endoprosthetic device cannulated by a guidewire;

FIG. 8 illustrates a perspective view of a fenestration in an endoprosthetic device cannulated by a catheter;

FIG. 9 illustrates a perspective view of an endoprosthetic device coupled to a branch endoprosthetic device.

FIG. 10 illustrates a perspective view of an endoprosthetic device comprising a curvilinear fenestration;

FIG. 11 illustrates a perspective view of an endoprosthetic device comprising a patch having a curvilinear edge;

FIG. 12A illustrates a cross-sectional view of a folded or pleated patch in a flattened configuration;

FIG. 12B illustrates a cross-sectional view of a folded or pleated patch in a non-flattened configuration;

FIG. 13 illustrates a perspective view of an endoprosthetic device comprising a patch having protuberances capable of detection by a guidewire;

FIG. 14 illustrates a cross-sectional view of a patch comprising a protuberance capable of detection by a guidewire;

FIG. 15A illustrates a perspective view of a constrained endoprosthetic device precannulated by a guidewire;

FIG. 15B illustrates a perspective view of an unconstrained endoprosthetic device precannulated by a guidewire;

FIG. 15C illustrates a perspective view of an unconstrained endoprosthetic device comprising a fenestration cannulated by a guidewire;

FIG. 16A illustrates a perspective view of a constrained endoprosthetic device precannulated by a first removable guidewire tube and a second removable guidewire tube; and

FIG. 16B illustrates a perspective view of an unconstrained endoprosthetic device precannulated by a first guidewire and a second guidewire.

### DETAILED DESCRIPTION OF THE ILLUSTRATED EMBODIMENTS

Persons skilled in the art will readily appreciate that various aspects of the present disclosure may be realized by any number of methods and apparatuses configured to perform the intended functions. Stated differently, other methods and apparatuses may be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

The terms “branch vessel,” “branch,” “side-branch” and/or “side-branch vessel” can refer to a vessel that branches from a main or otherwise primary vessel. Likewise, a main or primary vessel can refer to any vessel from which a branch vessel branches.

The terms “endoprosthetic device” and “endoprosthesis” can refer to any medical device capable of being implanted and/or deployed within a body lumen. A “main endoprosthetic device” and/or a “main endoprosthesis” can refer to any medical device capable of being deployed within a first or main body lumen. A “branch endoprosthetic device” or “branch endoprosthesis” can refer, in like manner, to any medical device capable of being deployed within a second or branch body lumen. A branch endoprosthetic device can, in various embodiments, and as described herein, be coupled to a main endoprosthetic device.

Throughout this specification and in the claims, the term “proximal” refers to a location that is, or a portion of an endoprosthetic device that when implanted is, closer to the heart or a similar anatomical reference point. Similarly, the term “proximally” refers to a direction towards the heart or other reference point. Within a vessel or other body lumen, a movement may be in a proximal direction if such movement within the vessel or lumen would lead to the heart or other reference point.

Similarly, the term “distal” refers to a location that is, or a portion of an endoprosthetic device that when implanted is, farther from the heart or other reference point. Likewise, the term “distally” refers to a direction away from the heart or other reference point. Within a vessel or other body lumen, a movement may be in a distal direction if such movement within the vessel or lumen leads away from the heart or other reference point.

Notwithstanding the foregoing, since the present disclosure is not limited to peripheral or central approaches, the device should not be narrowly construed when using the terms proximal or distal since device features may be slightly altered relative to the anatomical features and the device position relative thereto.

As used herein, the term “constrain” may mean (i) to limit expansion, occurring either through self-expansion or expansion assisted by a device, of the diameter of an expandable implant, or (ii) to cover or surround, but not otherwise restrain, an expandable implant (e.g., for storage or biocompatibility reasons and/or to provide protection to the expandable implant and/or the vasculature).

While specific embodiments are described in greater detail below, in general, the present disclosure will focus primarily upon devices and methods for treating a body lumen, such as a blood vessel. In various embodiments, a main endoprosthetic device can comprise a side opening or fenestration, such as a slit or incision. The fenestration can be capable of two configurations: a first, closed, fluid impermeable or semi fluid impermeable configuration, and a second, open configuration.

The main endoprosthetic device can further comprise a layer of material coupled to an inner surface of the device. This layer can be referred to herein, as a “patch” or “support wall.” In various embodiments, the patch can cover the fenestration. Further, the patch can have a first portion and a second portion, both of which can receive, in various embodiments, a branch endoprosthetic device. These portions can comprise end or edge portions and can enable antegrade and/or retrograde flow between the main endoprosthetic device and the branch endoprosthetic device.

In various embodiments, the branch endoprosthetic device can be received by the first open end and/or the second open end of the patch and coupled to the main endoprosthetic device between the patch and an inner surface of the main endoprosthetic device. The patch can be distensible and can exert a pressure against the branch endoprosthetic device to couple or hold the branch device against the main endoprosthetic device within a support channel defined by a separation between the patch and the inner surface of the main endoprosthetic device. The branch endoprosthetic device can further exit or extend through the main endoprosthetic device at the fenestration.

Therefore, with reference now to FIG. 1, an endoprosthesis or endoprosthetic device 100 is shown. The endoprosthetic device 100 can be deployed within a main or primary vessel. In various embodiments, the endoprosthetic device 100 can comprise a tube or a substantially tubular lumen. The tube or tubular lumen can include a first end 102 and/or a second end 104, and a side wall 106 can extend between the first end 102 and the second end 104. The side wall 106 can comprise an inner surface that defines a fluid flow channel. Thus, where the endoprosthetic device comprises a main endoprosthetic device, the fluid flow channel or tubular lumen can be a main fluid flow channel or lumen.

The endoprosthetic device 100 can further include a side opening, fenestration, or fenestrated portion 114. The fenestration 114 can comprise an opening, a slit, an incision, and the like. Further, in various embodiments, the fenestration 114 can comprise a weakened area and/or portion. Such a weakened portion can be perforated by a plurality of holes, scores, and the like. Similarly, a weakened portion can, relative to other portions of the main endoprosthetic device 100, comprise a thinner or less dense surface or material. Likewise, in various embodiments, a fenestration 114 can comprise a seal that can be broken (e.g., by a guidewire and/or a catheter).

The fenestration 114 can assume a variety of configurations. For example, the fenestration 114 can assume a closed configuration, in which the fenestration 114 remains substantially closed and/or fluid impermeable and/or substantially or semi fluid impermeable. In addition, the fenestration 114 can assume an open configuration, as shown, for example, at FIGS. 2 and 3, in which the fenestration is opened by a branch endoprosthetic device 202. In various embodiments, the fenestration 114 can be incrementally, partially, and/or selectively opened and/or closed. For example, the fenestration 114 can be opened to a first extent to accommodate or receive a branch endoprosthetic device having a first diameter. Likewise, the fenestration 114 can be opened to a second extent to accommodate or receive a branch endoprosthetic device having a second diameter.

The endoprosthetic device 100 can also comprise a layer of material such as a “support wall” or “patch” 108. The patch 108 can comprise a graft material, such as PTFE and/or ePTFE. The patch can be coupled to and/or disposed along an inner or luminal surface of the side wall 106. For example, the patch 108 can be joined or coupled to the side wall 106 along one or more of its longitudinal edges 110a and/or 110b. The patch may be variously joined to the side wall 106, e.g., via an adhesive substance, such as FEP or stitching. However, in various embodiments, the patch 108 is not joined to the side wall 106 along one or more of its latitudinal edges 112a and/or 112b. Further, in various embodiments, the patch 108 is not joined to the side wall 106 within a region interior to the edges 110a and 110b. Thus, the patch 108 can, together with the side wall 106, form a channel or pocket. Further, although the patch 108 is described herein with reference to one or more edges, in various embodiments, the patch 108 can comprise any shape.

The channel can, in certain embodiments, be flexible and/or distensible. For instance, the patch 108 can be distensible or movable between a closed configuration adjacent to the inner surface of the side wall 106 and an open configuration spaced apart from the inner surface of the side wall 106. In the open configuration, a channel (e.g., a support channel) can be formed between the inner surface of the side wall 106 and the patch 108. In various embodiments, the side wall 106 can also be somewhat flexible and/or distensible. As shown, the patch 108 can further overlap or cover the fenestration 114.

With reference to FIGS. 2 and 3A, branch endoprostheses are shown coupled to main endoprostheses in two configurations. Specifically, a branch endoprosthetic device 202 can be coupled to a main endoprosthetic device 100 in a first configuration (e.g., see FIG. 2) and/or a second configuration (e.g., see FIG. 3A). In either of these configurations, fluid may flow from the main endoprosthetic device 100 into the branch endoprosthetic device 202 with, or against, a direction of fluid flow within the main device 100. Thus, fluid may be allowed to flow from the main device 100 and into the branch device 202, or vice versa, in either an antegrade or retrograde direction.

To this end, a patch 108 can include one or more open portions, such as one or more open ends or edges. These open ends can be capable of receiving a branch endoprosthesis 202, and each end can act as an opening to the support channel, through which the branch endoprosthetic device can be coupled to the main endoprosthetic device. For example, with particular attention to FIG. 2, an open end 210 can permit a branch endoprosthesis to enter a support channel in the first configuration. Similarly, with particular attention to FIG. 3A, an open end 306 can permit a branch endoprosthesis to enter a support channel in the second configuration. Further, where a patch includes two open ends or edges 210 and 306, a branch endoprosthetic device can be implanted within a body lumen and coupled, in situ, to a main endoprosthetic device in either of the first and/or second configuration.

Thus, a branch endoprosthetic device 202 can be coupled to a main endoprosthetic device 100 via a support channel and exit a support channel at a fenestration 114 made in a side wall of the main endoprosthetic device 100. Moreover, as described herein, a fenestration 114 can remain substantially closed and/or fluid impermeable and/or substantially or semi fluid impermeable when the fenestration 114 is not opened by a branch endoprosthetic device 202. The patch 108 can further reduce and/or eliminate fluid flow between the fenestration 114 and the patch 108 when the patch 108 is in a closed configuration adjacent to the inner surface of the side wall 106. In addition, in various embodiments, a main endoprosthetic device 100 can include a plurality of fenestrations and/or a plurality of patches, each of which may couple a branch endoprosthetic device 202 to the main endoprosthetic 100. Thus, in various embodiments, a main endoprosthetic device 100 can be coupled to numerous branch endoprosthetic devices 202.

Fluid flow between the fenestration 114 and the patch 108 can be further reduced and/or eliminated by the introduction of a bond between at least a portion of the patch 108 and an inner surface of the side wall 106. Any suitable method can be used to form the bond between the patch 108 and the inner surface of the side wall 106. For example, the bond can be formed through the use of one or more adhesives, a heat bonding process, stitching, and the like. Adhesives can include any material suitable to the purpose, including, for example, FEP, PATT, and the like.

Thus, with attention to FIG. 3B, in certain embodiments, the entire patch 108 can be coupled to the inner surface of the side wall 106. Where this configuration is employed, a channel 302 of varying widths or dimensions can be opened, detached, or released between the patch 108 and the inner surface of the side wall 106 by a device such as a guidewire, catheter, balloon, or a branch endoprosthetic device. In particular, any of the aforementioned devices can break the bond between the patch 108 and the inner surface of the side wall 106.

Similarly, in various embodiments, the channel 302 can be pre-formed between the patch 108 and the inner surface of the side wall 106. For instance, the patch 108 can be joined to the side wall 106 except in the region comprising the channel 302. This configuration can permit easier access by a guidewire or catheter to the channel 302. In various embodiments, the channel 302 can comprise the support channel, as described herein, and/or the channel 302 can comprise a narrower precursor to the support channel 302, which can be opened or widened further by the insertion, for example, of a catheter and/or a branch endoprosthetic device 202 to form the support channel.

Turning to FIGS. 4 and 5, cross-sections of a branch endoprosthetic device 202 coupled to a main endoprosthetic device 100 in each of the first and second configurations are shown. The branch device 202 can extend into the main device 100 between the patch 108 and a portion of the side wall 406a-406b and/or 506a-506b of the main device 100. Thus, the patch 108 can form the support channel in combination with one or more portions of the side wall 406a-406b and/or 506a-506b. In addition, as described above, the support channel can distend to receive and couple the branch endoprosthetic device 202 to the main endoprosthetic device 100. In various embodiments, the branch endoprosthetic device 202 can extend within the lumen of the main endoprosthetic device 100 beyond the edge of the patch 108. Likewise, in various embodiments, the branch endoprosthetic device 202 can be substantially flush with the edge of the patch 108.

In operation, and with attention now to FIGS. 6-9, deployment can proceed as follows. A guidewire 602 can be deployed within a main endoprosthetic device 100 (which may be pre-deployed within a body lumen). As shown at FIG. 7, the guidewire 602 can be manipulated to locate a fenestration 114 made in the patch 108. The guidewire 602 can further open the fenestration 114, such that, as shown with reference to FIG. 8, a catheter 800 can follow the guidewire 602 through the open fenestration 114. In various embodiments, the guidewire 602 can locate the fenestration 114 through a visualization marker, such as a radiopaque marker, which can be included in any location on or within the main and/or branch endoprostheses. For example, in various embodiments, such a marker can be included on or proximate to the fenestration 114.

The catheter 800 can, in various embodiments, include or deliver a branch endoprosthetic device 202. The branch endoprosthetic device 202 can comprise an expandable (e.g., a balloon expandable) endoprosthesis and/or a self-expanding endoprosthesis. Further, the branch endoprosthetic device 202 can expand or deploy from a constrained diameter to an unconstrained diameter, as shown, while it is on the catheter 800. Thus, the branch endoprosthetic device 202 can expand within the distensible support channel, such that the branch device 202 is coupled, between the patch 108 and an inner surface of the side wall of the main endoprosthetic device 100, to the main endoprosthetic device 100. The deployed branch endoprosthetic device 202 can further expand within the fenestration 114, such that a relatively close (e.g., fluid impermeable and/or substantially fluid impermeable) seal is made between the branch device 202 and the main device 100. A deployed branch endoprosthetic device 202 is shown at FIG. 9.

In various embodiments, as depicted at FIG. 9, some open space, or a first “gutter” 904a may remain, proximate to the fenestration, between the patch 108 and the side wall of the main endoprosthetic device 100. A second gutter 904b may exist at an opposite side of the fenestration. Likewise third and fourth gutters 908a and 908b may be formed between the patch 108 and the side wall of the main endoprosthetic device 100. These gutters 908a and 908b may, as shown, be formed within the main fluid flow channel of the main endoprosthetic device 100. In other words, the third and fourth gutters 908a and 908b may be formed proximate to an open end of the patch 108.

In some embodiments, one or more of these gutters can be reduced, eliminated, and/or occluded by a branch endoprosthetic device 202. This can be accomplished by selecting one or more desired branch endoprosthetic device 202 shapes and/or materials which can fully or partially block or occlude one or more gutters. Thus, for example, a branch endoprosthetic device 202 can comprise a conformable material, such as a graft material, which is capable of conforming to fit within one or more of the gutters. Similarly, in various embodiments, one or more gutters can be reduced, eliminated, and/or occluded by a branch endoprosthetic device 202 that is preformed to fit within one or more of the gutters. Such a branch endoprosthetic device 202 can comprise, for example, a pinched or ellipsoidal shape. In addition, in various embodiments, a gutter can be reduced or eliminated by the inclusion of a strip of material (e.g., a thread or fiber) within or along the fenestration 114 and/or the within or along one of more of the open ends of the patch 108. These strips of material can be tightened or cinched to reduce or eliminate one or more gutters. Further, in various embodiments, a gutter can be reduced or occluded by utilizing a material, such as a flexible or compliant material (e.g., ePTFE) and/or a distensible and/or elastomeric material (e.g., any polymer and/or fluoropolymer, such as those described herein, and/or any fluoroelastomer and/or polyurethane), that is capable of conforming to encircle or ensconce the branch endoprosthetic device such that no gutters are formed and/or such that gutters are substantially reduced.

In addition, and with reference to FIG. 10, the first and second gutters 904a and 904b can be reduced or eliminated by the use of a curvilinear or crescent-shaped fenestration 1002. This fenestration 1002 can be formed, as described above, in a side wall of the main endoprosthetic device 100. In addition, the fenestration 1002 can, depending upon the configuration in which the branch endoprosthetic device is coupled to the main endoprosthetic device 100 (e.g., the first or second configuration, as described above), comprise a convex and/or concave fenestration.

Similarly, and with reference to FIG. 11, the third and fourth gutters 908a and 908b can be reduced or eliminated by the use of a curvilinear or crescent-shaped open end 1102. This curvilinear open end 1102 can be formed, as described above, in a patch 108. In addition, the curvilinear open end 1102 can, depending upon the configuration in which the branch endoprosthetic device is coupled to the main endoprosthetic device 100 (e.g., the first or second configuration, as described above), comprise a convex and/or concave open end 1102.

Further, with attention to FIGS. 12A and 12B, one or more gutters can be reduced or eliminated by folding or pleating the patch 108. For example, as shown at FIG. 12A, where an endoprosthetic device 100 is constrained, a pleat 1202 made in the patch 108 can lie substantially flat against or flush with the inner surface of the side wall 106 of the device 100. Thus, in a flattened configuration, the pleated patch 108 can, as described above, comprise a fluid impermeable or substantially fluid impermeable barrier to fluid flow through the fenestration 114.

However, as shown at FIG. 12B, as the endoprosthetic device 100 expands from a constrained diameter to an unconstrained diameter, the pleat 1202 can enlarge or balloon to form a support channel 1204 with the inner surface of the side wall 106. The support channel 1204, formed in this manner, can aid in the occlusion of one or more gutters (e.g., the gutters 908a and 908b) by more completely encircling or ensconcing the branch endoprosthetic device 202. The support channel 1204 can further aid, as described herein, with location (e.g., by a guidewire) of the support channel 1204. Further still, as the channel 1204 can be somewhat preformed in this example, a branch endoprosthetic device 202 can more easily deploy within the support channel 1204; that is, because the branch device 202 may not, through its own implantation and expansion process, distend the side wall to form the channel 1204.

With reference to FIGS. 13 and 14, a patch 108 can include one or more features capable of aiding detection of the patch 108. These features can comprise materials of varying hardness, texture, shape, and the like. Specifically, in various embodiments, these features can comprise one or more convexities or protuberances 1304 and/or 1306. A protuberance 1304 and/or 1306 can be capable of detection by a guidewire. In other words, a protuberance 1304 and/or 1306 can comprise a guidewire guide. Thus, a protuberance 1304 and/or 1306 can facilitate cannulation, by a guidewire, of the patch 108 and/or the fenestration 114 in the side wall of the main endoprosthetic device 100. FIG. 13 shows a cross-sectional view of a protuberance 1304 and/or 1306. In various embodiments, as shown, a protuberance 1402 can be disposed opposite a concave portion or concavity 1404. A guidewire can, where such a concavity 1404 is included in a patch, be used to detect the concavity 1404, which can permit the guidewire easier access between the patch and the inner surface of the side wall of the main endoprosthetic device. More particularly, in certain embodiments, the guidewire can be advanced beneath the concavity 1404 to cannulate the support channel. Further, in various embodiments, the protuberance 1402 and/or the concavity 1404 can include a visualization element, such as, for example, a radiopaque marker and/or any other marker or material capable of detection.

In various embodiments, an endoprosthetic device 100 can be precannulated (that is, cannulated prior to implantation or deployment within a body lumen) by a variety of methods. For example, as shown with reference to FIG. 15A, an endoprosthetic device 100 can be precannulated by a first guidewire 1502 and a second guidewire 1504. As described above, the endoprosthetic device 100 can be advanced, during deployment, along a catheter 1506 that follows the first guidewire 1502. The second guidewire 1504 can be coupled to or constrained (e.g., as by a constraining sheath) within a lumen of the endoprosthetic device 100 and through the space between the patch 108 and the side wall 106 of the endoprosthetic device 100. The second guidewire 1504 may thus terminate at a point beyond the endoprosthetic device 100. For instance, as shown, the second guidewire 1504 can terminate proximate to the catheter tip 1508. However, in various embodiments, the second guidewire 1504 can terminate at any position, including positions that are not beyond the catheter tip 1508, such as, for example, within the lumen of the constrained endoprosthetic device 100.

During deployment, and as shown now with reference to FIGS. 15B and 15C, the constrained endoprosthetic device 100 can expand to an unconstrained or deployed diameter. At this stage, the second guidewire 1504 can be free to move within the lumen of the endoprosthetic device 100 and through the space between the patch 108 and the side wall 106 of the endoprosthetic device 100. Thus, a physician can, for example, advance and/or retract the second guidewire within the lumen of the endoprosthetic device 100. More particularly, as shown at FIG. 15C and as described herein, the physician can manipulate the second guidewire 1504 to locate and/or cannulate the fenestration 114.

Similarly, in various embodiments, a fenestration 114 can itself be precannulated. For instance, as shown with reference to FIGS. 16A and 16B, a fenestration 114 can, prior to implantation within a body lumen, be precannulated by one or more guidewire tubes, e.g., a first guidewire tube 1602 and/or a second guidewire tube 1604. A guidewire tube can comprise a cannula and/or any other tubular structure capable of receiving a guidewire. Guidewire tubes are described within U.S. Pat. No. 8,273,115 to Hamer et al., issued Sep. 25, 2012, entitled “Side branched endoluminal prostheses and methods of delivery thereof,” which is hereby incorporated by reference in its entirety. A guidewire tube can be fixed or held in place within a lumen of a constrained endoprosthetic device, as shown, for example, with reference to FIG. 16A. And, in various embodiments, a fenestration 114 can also be precannulated by one or more guidewires.

Prior to and/or during deployment, as shown with continuing reference to FIGS. 16A and 16B, a guidewire 1608 and/or 1606 can be advanced within the lumen of the guidewire tube 1602 and/or the lumen of the guidewire tube 1604, respectively. The guidewire tubes 1602 and 1604 can be open at either or both of a distal and/or a proximal end. Thus, prior to and/or during deployment, either or both of the guidewire tubes 1602 and/or 1604 can be removed or retracted to expose the guidewire 1608 and/or 1606 situated within. The fenestration 114 can thus, through the use of the precannulated guidewire tubes 1602 and 1604, be cannulated by one or more guidewires 1608 and/or 1606. Where the fenestration 114 is precannulated in this way, it may not be necessary to locate the fenestration 114 during deployment. In addition, and as described above, this method can facilitate the implantation of a branch endoprosthetic device 202 in either or both of a first and/or second configuration (e.g., either of an antegrade and/or retrograde configuration). Further, in various embodiments, a guidewire tube 1602 and/or 1604 can be closed at a proximal or distal end to ensure that the tube is not inadvertently left within a body lumen after an endoprosthesis implantation procedure is complete.

A graft comprising any of the grafts and/or stent-grafts described above can be made up of any material which is suitable for use as a graft in the chosen body lumen. A graft can comprise one or a variety of materials. Furthermore, a graft can comprise multiple layers of material, which can be the same material or different material. Although a graft can have several layers of material, the graft can have a layer that is formed into a tube (innermost tube) and an outermost layer that is formed into a tube (outermost tube). In some embodiments, a graft can be fenestrated in-situ with a fenestration tool.

Many graft materials are known, and in various embodiments, these materials can be used in combination and assembled together to comprise a graft as well as a patch. These materials may be further extruded, coated and/or formed from wrapped films, and/or a combination thereof. Polymeric materials, biodegradable materials, and/or natural materials can be used for specific applications.

In various embodiments, a graft and/or patch can comprise synthetic polymers including nylon, polyacrylamide, polycarbonate, polyformaldehyde, polymethylmethacrylate, polytetrafluoroethylene, polytrifluorochlorethylene, polyvinylchloride, polyurethane, elastomeric organosilicon polymers, polyethylene, polypropylene, polyurethane, polyglycolic acid, polyesters, polyamides, their mixtures, blends, and copolymers. In a variety of embodiments, a graft can be made from a class of polyesters such as polyethylene terephthalate including DACRON® and MYLAR® and polyaramids such as KEVLAR®, polyfluorocarbons such as polytetrafluoroethylene (PTFE) with and without copolymerized hexafluoropropylene (TEFLON® or GORE-TEX®), and porous or nonporous polyurethanes. Further, in a variety of embodiments, a graft can comprise expanded fluorocarbon polymers (especially PTFE), materials.

In various embodiments, fluoropolymers can include polytetrafluoroethylene (PTFE), expanded PTFE (ePTFE), fluorinated ethylene propylene (FEP), copolymers of tetrafluoroethylene (TFE) and perfluoro (propyl vinyl ether) (PEA and/or PPVE), perfluoro ethyl vinyl ether (PEVE), perfluoro methy vinyl ether (PMVE), homopolymers of polychlorotrifluoroethylene (PCTFE), and its copolymers with TFE, ethylene-chlorotrifluoroethylene (ECTFE), copolymers of ethylene-tetrafluoroethylene (ETFE), polyvinylidene fluoride (PVDF), and polyvinylfluoride (PVF). In various embodiments, a graft and/or patch can comprise any combination of the materials listed above. Further, in various embodiments, a graft and/or patch can be substantially impermeable and/or permeable to bodily fluids. A substantially impermeable graft or patch can be made from materials that are substantially impermeable to bodily fluids or can be constructed from permeable materials treated or manufactured to be substantially impermeable to bodily fluids (e.g. by layering different types of materials described above or known in the art). In various embodiments, a stent-graft and/or a side-branch stent-graft, as described above, can be made from any combination of the materials described above, including ePTFE.

Any stent, including stent and/or stent members can be generally cylindrical when restrained and/or when unrestrained and may comprise helically arranged undulations having a plurality of helical turns. In a variety of embodiments, undulations can be aligned so that they are “in-phase” with each other. More specifically, undulations can comprise apices in opposing first and second directions. When these undulations are in-phase, apices in adjacent helical turns are aligned so that apices can be displaced into respective apices of a corresponding undulation in an adjacent helical turn. In certain embodiments, undulations can have a sinusoidal shape, a U shape, a V shape, and/or an ovaloid shape.

In various embodiments, a stent can be fabricated from a variety of biocompatible materials including commonly known materials (or combinations of materials) used in the manufacture of implantable medical devices. Such materials can include 316L stainless steel, cobalt-chromium-nickel-molybdenum-iron alloy (“cobalt-chromium”), other cobalt alloys such as L605, tantalum, nitinol, or other biocompatible metals. In some embodiments, any stent and/or stent-graft described herein can comprise a balloon expandable stent and/or stent-graft and/or a self-expanding stent and/or stent-graft. Further, in certain embodiments, a stent can comprise a wire wound stent, which may or may not comprise undulations.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications can be made, especially in matters of structure, materials, elements, components, shape, size, and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

What is claimed is:

1. An endoprosthetic device comprising:
a tube comprising a first end and a second end, the tube further comprising a side wall extending between the first end and the second end, the side wall comprising an inner surface defining a main fluid flow channel, the side wall fenestrated by a side opening;
a support wall disposed along an inner surface of the side wall, the support wall overlapping the side opening, the support wall being movable between a closed configuration adjacent to the inner surface and an open configuration spaced apart from the inner surface, the open configuration defining a support channel,
wherein the support wall is configured to support a branch endoprosthetic device extending through at least one of the support channel and the side opening such that the branch endoprosthetic device is in fluid communication with the main fluid flow channel.

2. The endoprosthetic device of claim 1, the support channel comprising a first open end configured to permit antegrade flow, the support channel further comprising a second open end configured to permit retrograde flow.

3. The endoprosthetic device of claim 1, wherein the side opening comprises a slit.

4. The endoprosthetic device of claim 3, wherein the slit includes a radiopaque marker.

5. The endoprosthetic device of claim 3, wherein the slit is curvilinear.

6. The endoprosthetic device of claim 3, wherein the slit is crescent-shaped.

7. The endoprosthetic device of claim 1, wherein the support wall extends circumferentially about at least a portion of the inner surface of the side wall.

8. The endoprosthetic device of claim 1, wherein the support wall is fixedly secured to the inner surface of the side wall.

9. The endoprosthetic device of claim 8, wherein the support wall is secured by an adhesive to the inner surface of the side wall.

10. The endoprosthetic device of claim 1, wherein the support wall is releasably coupled to the inner surface of the side wall.

11. The endoprosthetic device of claim 1, wherein the support wall is distensible.

12. The endoprosthetic device of claim 1, wherein the support wall is pleated.

13. The endoprosthetic device of claim 1, wherein the side wall is distensible.

14. The endoprosthetic device of claim 1, the side wall comprising a guidewire guide.

15. The endoprosthetic device of claim 14, the guidewire guide comprising a radiopaque marker.

16. The endoprosthetic device of claim 1, wherein the support wall comprises a curvilinear edge.

17. The endoprosthetic device of claim 1, wherein the branch endoprosthetic device comprises a conformable material, whereby the branch endoprosthetic device is capable of conforming to occlude at least a portion of a gutter formed between the side wall and the support wall.

18. The endoprosthetic device of claim 1, wherein the branch endoprosthetic device is preformed to occlude at least a portion of a gutter formed between the side wall and the support wall.

19. The endoprosthetic device of claim 1, wherein the endoprosthetic device is precannulated by a first guidewire and a second guidewire.

20. The endoprosthetic device of claim 1, wherein side opening is precannulated by a guidewire tube.

21. An endoprosthesis comprising:
a stent-graft, the stent-graft defining a substantially tubular primary lumen, the stent-graft further comprising a side wall, the side wall capable of forming an opening; and
a patch coupled to a luminal surface of the side wall, the patch comprising a first edge and a second edge, both the first edge and the second edge capable of receiving a branch endoprosthesis.

22. The endoprosthesis of claim 21, the side wall comprising a fenestrated portion, the fenestrated portion capable of forming the opening.

23. The endoprosthesis of claim 22, the fenestrated portion comprising a slit.

24. The endoprosthesis of claim 22, the patch covering the fenestrated portion to reduce fluid communication between the fenestrated portion and a branch vessel.

25. The endoprosthesis of claim 21, the patch being distensible to receive the branch endoprosthesis.

26. The endoprosthesis of claim 21, the side wall being distensible about the fenestrated portion to receive the branch endoprosthesis.

27. The endoprosthesis of claim 21, at least one of the patch and the side wall including a radiopaque marker.

28. The endoprosthesis of claim 21, the patch including a concave portion that is capable of being sensed by a guidewire.

29. The endoprosthesis of claim 21, the patch including a protuberance at one of the first edge and the second edge.

30. The endoprosthesis of claim 21, the patch comprising a curvilinear edge.

31. The endoprosthesis of claim 21, the branch endoprosthesis comprising a conformable material, whereby the branch endoprosthesis is capable of conforming to occlude at least a portion of a gutter formed between the side wall and the patch.

32. The endoprosthesis of claim 21, wherein the branch endoprosthesis is preformed to occlude at least a portion of a gutter formed between the side wall and the patch.

33. A method comprising:
deploying a main endoprosthesis within a body lumen;
deploying a guidewire within the main endoprosthesis to locate a patch coupled to a luminal surface of the main endoprosthesis, the patch covering a fenestrated portion of the main endoprosthesis; and
deploying a branch endoprosthesis within the main endoprosthesis such that the branch endoprosthesis extends between the patch and the luminal surface of the main endoprosthesis and exits from the main endoprosthesis at the fenestrated portion.

34. The method of claim 33, the patch comprising a protuberance for guiding a guidewire.

35. The method of claim 33, at least one of the fenestrated portion and the patch including a radiopaque marker.

36. The method of claim 33, the fenestrated portion comprising a slit.

37. The method of claim 33, the patch comprising first open edge and a second open edge, both the first open edge and the second open edge capable of receiving the branch endoprosthesis to facilitate either of an antegrade flow or a retrograde flow.

38. The method of claim 33, wherein the patch is distensible.

39. The method of claim 33, wherein the luminal surface is distensible.

40. The method of claim 33, wherein the fenestrated portion is curvilinear.

41. The method of claim 33, the patch comprising a curvilinear edge.

42. The method of claim 33, the branch endoprosthesis comprising a conformable material, whereby the branch endoprosthesis is capable of conforming to occlude at least a portion of a gutter formed between the luminal surface of the main endoprosthesis and the patch.

43. The method of claim 33, wherein the branch endoprosthesis is preformed to occlude at least a portion of a gutter formed between the luminal surface of the main endoprosthesis and the patch.
